(19) United States
(12) Patent Application Publication
(10) Pub. No.: US 2015/0359465 A1
(43) Pub. Date: Dec. 17, 2015

(54) HAND HYGIENE MOMENT MEASURING SYSTEM

(71) Applicant: MARUISHI PHARMACEUTICAL CO., LTD., Osaka-shi, Osaka (JP)

(72) Inventor: Yuji Wada, Takatsuki-shi, Osaka (JP)

(73) Assignee: Maruishi Pharmaceutical Co., Ltd., Osaka-shi, Osaka (JP)

(21) Appl. No.: 14/764,498

(22) PCT Filed: Jan. 31, 2014
(86) PCT No.: PCT/JP2014/052812

§ 371 Date: Jul. 29, 2015

(30) Foreign Application Priority Data
Feb. 1, 2013 (JP) 2013-018442

(51) Int. Cl. A61B 5/11; G01B 21/00

(52) U.S. Cl. CPC A61B 5/1116; G01B 21/00

(57) ABSTRACT

The hand hygiene moment measuring system of the present invention includes a subject sensing device (2) to sense a subject's position and posture, a movement sensing device (3) to sense a subject's movement, a storage unit (4) to store particular combinations corresponding to hand hygiene opportunities, and a counter (5) to count a number of times combinations of a sensing information of the subject sensing device, a sensing information of the movement sensing device, and an object position information indicating a position of an object, achieved the combinations stored in the storage unit.

### TECHNICAL FIELD

The present invention relates to a hand hygiene moment measuring system.

### BACKGROUND ART

In recent years, a further suppression of Healthcare-associated infections (HAIs) is required at medical settings. It is known that HAIs can be effectively suppressed by proper hand hygiene, e.g., washing hands under running water using soap, or rubbing hands using an alcohol based antiseptics.

For example, guidelines for hand hygiene of United States CDC (United States Centers for Disease Control and Prevention) or WHO (World Health Organization) disclose that an increase in hand hygiene compliance rates (i.e., the number of taken hand hygiene actions/the number of occurrence of events requiring hand hygiene) reduces rates of HAIs. Note that, it is reported that, with the hand hygiene compliance rates of 70% or more, the MRSA infection rates has successfully dropped to about 10% or less.

There are provided some apparatuses that measure numerator data (the number of taken hand hygiene actions), out of data pieces serving as indexes in planning an improvement in the hand hygiene compliance rates. For example, there are provided apparatuses that indirectly measure numerator data by measuring the used amount of antiseptics or measuring the number of times a dispenser was pushed.

Further, Patent Literature 1 (JP-A-No. 2009-282442) discloses an apparatus that recognizes a subject's action via movement detecting device attached to the subject. The apparatus determines whether or not a hand hygiene action is required and evaluates the action status based on the recognition result. Then, the apparatus automatically provides advice to the subject in accordance with the evaluation result.

### CITATION LIST

### Patent Literature

PTL 1: JP-A-No. 2009-282442

### Non Patent Literature

NPL 1: Centers for Disease Control and Prevention, Guideline for Hand Hygiene in Health-Care Settings, translated by Takashi OKUBO, translation supervised by Hiroyoshi KOBAYASHI, MEDICUS SHUPPAN, Publishers Co., Ltd., Osaka (2003)
NPL 2: WHO Guidelines on hygiene in health care (http:/whqlibdoc.who.int/publications/2009/9789241597906_eng.pdf)

### SUMMARY OF THE INVENTION

However, it is considered that, out of data pieces serving as indexes in planning an improvement in hand hygiene compliance rates, denominator data (i.e., the number of occurrence of events requiring hand hygiene, which is hereinafter referred to as hand hygiene opportunities) is difficult to indirectly measure, and that it is best to have an expert directly observe the subject to carry out measurement.

By an expert carrying out direct observation, the hand hygiene opportunities can be measured highly precisely. However, since the burden put on a person is very heavy in this method, WHO stipulates about a 20-minute session. Therefore, the hand hygiene opportunities throughout a day cannot be thoroughly measured. Further, since it is performed by a human, the determination as to whether or not it is a hand hygiene opportunity varies. Still further, the subject who is aware of being observed may take unusual actions only under observation. Thus, it is difficult to count ordinary hand hygiene opportunities.

On the other hand, with the conventional apparatuses such as the one disclosed in Patent Literature 1, the burden incurred by the measurement can be reduced because the measurement of the number of taken hand hygiene actions is automated. However, since the conventional apparatuses directly or indirectly measure the number of actually taken hand hygiene action, the hand hygiene opportunities cannot be measured highly precisely.

Even when the number of taken hand hygiene actions could be measured, when the hand hygiene opportunities are great in number, naturally the hand hygiene compliance rates are decreased. In this case, even when the number of taken hand hygiene actions is great, the rates of HAIs will not be decreased. Accordingly, in order to decrease the rates of HAIs, it is important to measure hand hygiene opportunities highly precisely.

Accordingly, an object of the present invention is to solve the issue noted above, and to provide a hand hygiene moment measuring system that can reduce the burden incurred by measurement of hand hygiene opportunities and that can carry out such measurement highly precisely.

In order to solve the above issue, a hand hygiene moment measuring system, including:

a subject sensing device to sense a subject's position and posture;

a movement sensing device to sense a subject's movement;

a storage unit to store particular combinations corresponding to hand hygiene opportunities; and

a counter to count a number of times combinations of a sensing information of the subject sensing device, a sensing information of the movement sensing device, and an object position information indicating a position of an object, achieved the combinations stored in the storage unit.

### Advantageous Effects of the Invention

With the hand hygiene moment measuring system of the present invention, thanks to the structure described above, the burden incurred by measurement of hand hygiene opportunities can be reduced and such measurement can be carried out highly precisely.

### BRIEF DESCRIPTION OF THE DRAWINGS

These aspects and features of the invention will be apparent from the following description concerning a preferred embodiment with respect to the accompanying drawings, in which:

FIG. 1 is a block diagram of a hand hygiene moment measuring system according to the first embodiment of the present invention.

FIG. 2 is a front view schematically showing a subject.

FIG. 3 is a plan view schematically showing a positional relationship among the object, the patient, and the subject when the subject is touching the patient.

FIG. 4 is a front view schematically showing a posture of the patient showed in FIG. 3.

FIG. 5 is a plan view schematically showing a positional relationship among the object, the patient, and the subject when changing an intravenous drip bag.

FIG. 6 is a front view schematically showing a posture of the patient shown in FIG. 5.

FIG. 7 is a plan view schematically showing a positional relationship among the object, the patient, and the subject at body fluid exposure risk.

FIG. 8 is a front view schematically showing a posture of the patient shown in FIG. 7.

FIG. 9 is a plan view schematically showing a positional relationship among the object, the patient, and the subject when the subject is touching any object in the patient's surrounding.

FIG. 10 is a diagram showing an example of information notified by the notifying unit.

FIG. 11 is a block diagram of a hand hygiene opportunity measuring system according to the second embodiment of the present invention.

FIG. 12 is a block diagram of a hand hygiene opportunity measuring system according to the third embodiment of the present invention.

### DESCRIPTION OF EMBODIMENTS

### Findings Forming a Basis of the Invention

The inventor of the present invention has made the following findings as a result of close study for achieving the object noted above.

Conventionally, measurement of hand hygiene opportunities by an expert is carried out by counting the following five phases generally listed in guidelines of WHO and the like (hereinafter referred to as five moments).

1. Before touching a patient

2. Before clean/aseptic procedures

3. At body fluid exposure risk

4. After touching a patient

5. After touching any object in the patient's surroundings

At these five moments, hand hygiene is required in order not to pass germs carried on the hands of a health-care worker to a patient or the like, or in order to remove germs carried on the hands by touching any contaminated environmental sites.

The inventor of the present invention has found that all the five moments can be sensed by sensing the position of any object such as a bed, the subject's position and posture, and the subject's movement, as a result of analysis of the movements of health-care workers at the five moments. Further, the inventor of the present invention has found that the burden incurred by measurement of hand hygiene opportunities can be largely reduced by automatically sensing the position of any object, the subject's position and posture, and the subject's movement by sensing device such as a sensor. Based on the foregoing, the inventor of the present invention has made the following invention.

According to a first aspect of the present invention, there is provided a hand hygiene moment measuring system, including:

a subject sensing device to sense a subject's position and posture;

a movement sensing device to sense a subject's movement;

a storage unit to store particular combinations corresponding to hand hygiene opportunities; and

a counter to count a number of times combinations of a sensing information of the subject sensing device, a sensing information of the movement sensing device, and an object position information indicating a position of an object, achieved the combinations stored in the storage unit.

According to a second aspect of the present invention, there is provided the hand hygiene moment measuring system according to the first aspect, further including

an object sensing device to sense the position of the object, wherein

the counter counts a number of times combinations of the sensing information of the subject sensing device, the sensing information of the movement sensing device, and a sensing information of the object sensing device, achieved the combinations stored in the storage unit.

According to a third aspect of the present invention, there is provided the hand hygiene moment measuring system according to the first aspect, further including

an input unit to receive inputs of the object position information, wherein

the storage unit stores the object position information input into the input unit, and

the counter counts a number of times combinations of the sensing information of the subject sensing device, the sensing information of the movement sensing device, and the object position information stored in the storage unit, achieved the combinations stored in the storage unit.

According to a fourth aspect of the present invention, there is provided the hand hygiene moment measuring system according to any one of the first to third aspects, wherein

the storage unit stores particular combinations corresponding to hand hygiene opportunities as being classified into a plurality of groups, and

the counter counts a number of times combinations of the sensing information of the subject sensing device, the sensing information of the movement sensing device, and the object position information stored in the storage unit, achieved the combinations stored in the storage unit with respect to each of the groups.

According to a fifth aspect of the present invention, there is provided the hand hygiene moment measuring system according to any one of the first to fourth aspects, further including a notifying unit to notify about the number of times counted by the counter.

According to a sixth aspect of the present invention, there is provided the hand hygiene moment measuring system according to any one of the first to fourth aspects, further including

a calculation unit to calculate the hand hygiene compliance rate based on the number of times counted by the counter, and

a notifying unit to notify the compliance rate calculated by the calculation unit.

Hereinafter, embodiments to the present invention will be described with reference to the drawings. It should be not that the present invention is not limited to these embodiments.

### First Embodiment

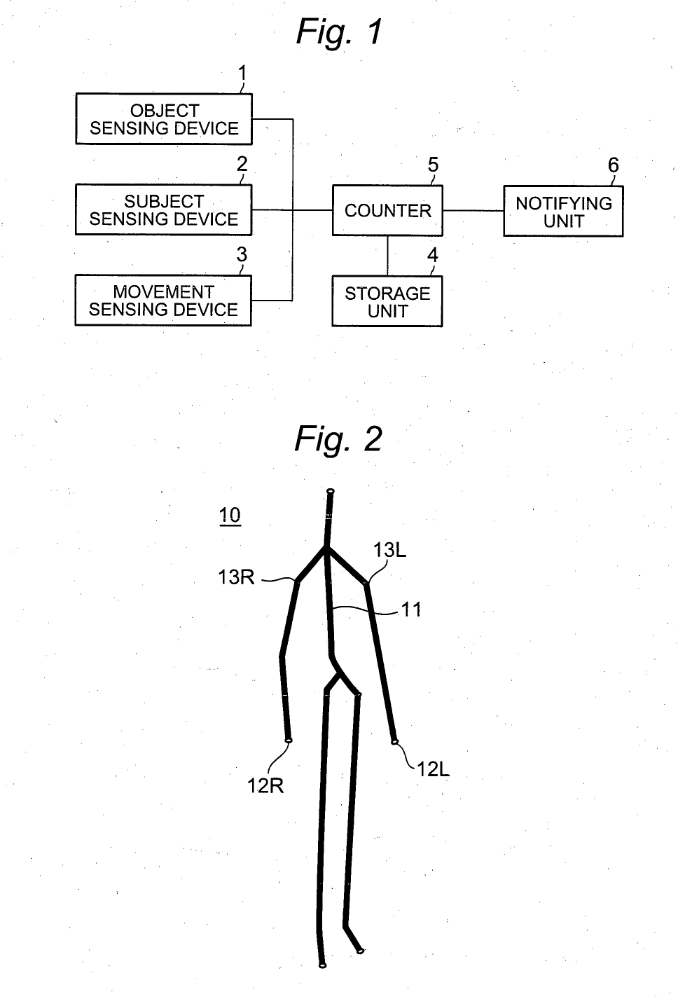

With reference to FIG. 1, a description will be given of the overall structure of a hand hygiene moment measuring system according to a first embodiment of the present invention. FIG. 1 is a block diagram of a hand hygiene moment measuring system according to the first embodiment.

In FIG. 1, the hand hygiene moment measuring system according to the first embodiment includes object sensing device 1, subject sensing device 2, movement sensing device 3, a storage unit 4, a counter 5, and a notifying unit 6.

The object sensing device 1 senses the position of an object. The “object” may be any item disposed in a hospital room being the area where hand hygiene opportunities are observed, the exemplary object including a bed, a bedside cabinet, a pillow, and an IV stand. The object sensing device 1 senses, for example, at what position (e.g., in three-dimensional coordinates) a bed is disposed in the hospital room.

The subject sensing device 2 senses the subject's position and posture. The “subject” is, for example, a health-care worker such as a doctor or a nurse. The subject sensing device 2 senses, for example, at what position (e.g., in three-dimensional coordinates) and in what posture (e.g., the forward-bent posture, the upright standing posture, the squatting posture, and the like) a nurse is present in the hospital room.

The movement sensing device 3 senses the subject's movement. The “subject's movement” is, for example, a shift (temporal change) of a particular site of the subject such as the right hand, the left hand, the trunk, and the like. The movement sensing device 3 senses, for example, the movement of the right hand when the health-care worker shifts his/her right hand from beside the bed to above the bed, for touching the patient. The movement sensing device 3 is different from the object sensing device 1 and the subject sensing device 2 in observing the shifting state of the subject, while sensing device 1 and 2 observe the object or the subject in the still state.

Note that, though the object sensing device 1, the subject sensing device 2, and the movement sensing device 3 are not particularly limited, it is preferable to use non-contact sensors. Thus, the work of attaching a sensor to the subject as in Patent Literature 1 can be eliminated, and usability can be largely improved. As the sensor having the functions of the sensing device 1 to 3, for example, “Kinect (registered trademark) for Windows” available from Microsoft Corporation may be employed. When such a sensor is employed as the sensing device 1 to 3, a plurality of (preferably, two) sensors should be provided, so as to eliminate any blind spot.

The storage unit 4 previously stores particular combinations corresponding to hand hygiene opportunities. In other words, the storage unit 4 previously stores the combinations of sensing information of the object sensing device 1, sensing information of the subject sensing device 2, and sensing information of the movement sensing device 3, which are indicative of hand hygiene opportunities. Particular combinations stored in the storage unit 4 will be detailed later.

The counter 5 counts the number of times combinations of the sensing information of the object sensing device 1, the sensing information of the subject sensing device 2, the sensing information of the movement sensing device 3 achieved the combinations stored in the storage unit 4.

The notifying unit 6 notifies the user about the number of times counted by the counter 5. The notifying unit 6 notifies, for example, the user about the number of times counted by the counter 5 visually by displaying the number on the display of a personal computer, or aurally by voice.

Note that, the storage unit 4, the counter 5, and the notifying unit 6 may be built in a personal computer.

Next, a description will be given of measurement of five moments with the hand hygiene moment measuring system according to the first embodiment.

In the first embodiment, though the subject sensing device 2 and the movement sensing device 3 are not particularly limited, the sensing device 2 and 3 recognize the subject as a stickman (a shape in which the sites including the joints of the subject are connected to each other by straight lines) as shown in FIG. 2, and perform their respective sensing functions.

Firstly, a description will be given of measurement of the hand hygiene opportunity “before touching a patient”.

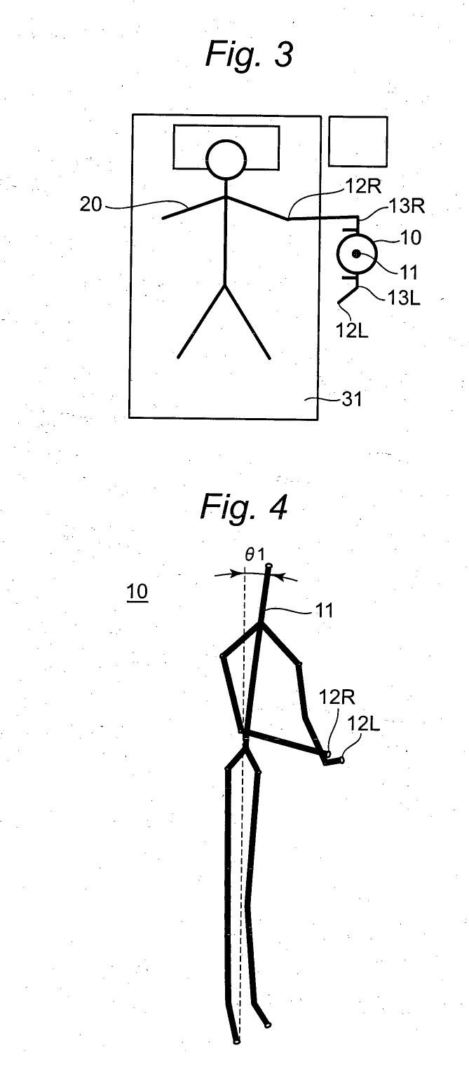

When a health-care worker touches a patient for performing medical practice, normally, his/her wrists come into the area above the bed, and he/she takes a slight forward-bent posture. FIGS. 3 and 4 show the state of a subject 10 touching a patient 20.

For example, when the subject sensing device 2 senses that a trunk 11 is inclined by a prescribed angle θ1 (e.g., 15 degrees) or more with reference to the vertical line, and when the movement sensing device 3 senses that a right wrist 12R or a left wrist 12L shifts above the position of a bed 31 sensed by the object sensing device 1, it is estimated that the subject 10 is touching the patient 20.

The storage unit 4 previously stores the combination of the sensing information pieces of the sensing device 1 to 3 at this time. The counter 5 counts the number of times the combination of the sensing information pieces of the sensing device 1 to 3 achieved the combination stored in the storage unit 4. Thus, the hand hygiene opportunity “before touching a patient” can be measured.

Note that, the “trunk” refers to the torso portion of the subject without the limbs and the head. It refers to the substantial central axis (the axis that extends in the extending direction of the spine) of the torso portion, assuming that the subject is a stickman as shown in FIG. 2.

Next, a description will be given of measurement of the hand hygiene opportunity “before clean/aseptic procedures”.

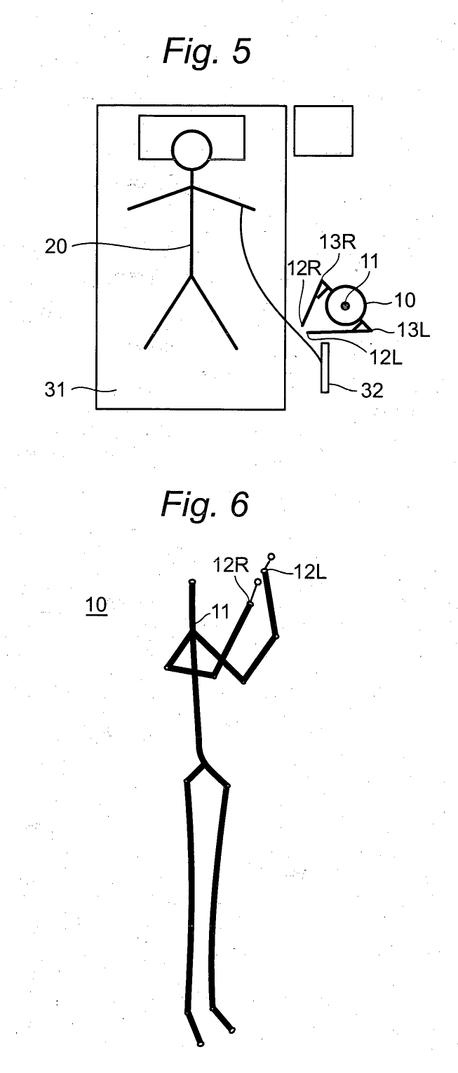

The “clean/aseptic procedures” refers to, for example, the procedure of changing an intravenous drip bag. When changing an intravenous drip bag, the health-care worker normally places both of his/her wrists in close proximity to each other near the height position of the intravenous drip bag, and turns his/her back to or stand sideways at the patient. FIGS. 5 and 6 show the state of the subject 10 changing an intravenous drip bag.

For example, when the subject sensing device 2 senses that the right wrist 12R and the left wrist 12L are positioned in the range in which they are away from each other by a prescribed distance (e.g., 30 cm) or less near the height position of an intravenous drip stand 32 sensed by the object sensing device 1, and that a straight line connecting between a right shoulder 13R and a left shoulder 13L are inclined by a prescribed angle (e.g., 45 degrees or more) with reference to the long side of the bed 31, it is estimated that the subject 10 is performing the procedure of changing an intravenous drip bag.

The storage unit 4 previously stores the combination of the sensing information pieces of the sensing device 1 to 3 at this time. The counter 5 counts the number of times the combination of the sensing information pieces of the sensing device 1 to 3 achieved the combination stored in the storage unit 4. Thus, the hand hygiene opportunity “before clean/aseptic procedures” can be measured.

Next, a description will be given of measurement of the hand hygiene opportunity “at body fluid exposure risk”.

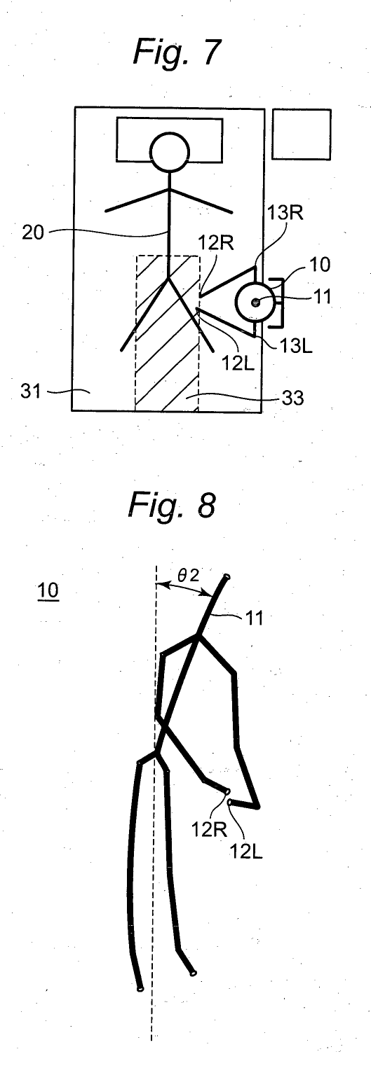

“At body fluid exposure risk” is, for example, when the health-care worker changes the diaper of a patient. When changing a diaper, the health-care worker normally places his/her wrists near the lower body of the patient, and takes a sharp forward-bent posture. FIGS. 7 and 8 show the state of the subject 10 changing a diaper.

For example, when the subject sensing device 2 senses that the right wrist 12R and the left wrist 12L are positioned in the range in which they are away from each other by a prescribed distance (e.g., 30 cm) or less above a prescribed region 33 of the bed 31 sensed by the object sensing device 1, and that the trunk 11 is inclined by a prescribed angle θ2 (e.g., 15 degrees) or more with reference to the vertical line, it is estimated that the subject 10 is changing a diaper.

The storage unit 4 previously stores the combination of the sensing information pieces of the sensing device 1 to 3 at this time. The counter 5 counts the number of times the combination of the sensing information pieces of the sensing device 1 to 3 achieved the combination stored in the storage unit 4. Thus, the hand hygiene opportunity “at body fluid exposure risk” can be measured.

Next, a description will be given of measurement of the hand hygiene opportunity “after touching a patient”.

After touching a patient, the health-care worker normally returns to the upright standing posture from the slight forward-bent posture, and leaves the bed.

For example, when the movement sensing device 3 senses that the trunk 11 returns to the substantially vertical state from the state being inclined by a prescribed angle θ1 or more with reference to the vertical line, and that the trunk 11 shifts by a prescribed distance from the area near the position of the bed 31 sensed by the object sensing device 1, it is estimated that the subject 10 has touched the patient 20.

The storage unit 4 previously stores the combination of the sensing information pieces of the sensing device 1 to 3 at this time. The counter 5 counts the number of times the combination of the sensing information pieces of the sensing device 1 to 3 achieved the combination stored in the storage unit 4. Thus, the hand hygiene opportunity “after touching a patient” can be measured.

Next, a description will be given of measurement of the hand hygiene opportunity “after touching any object in the patient's surroundings”.

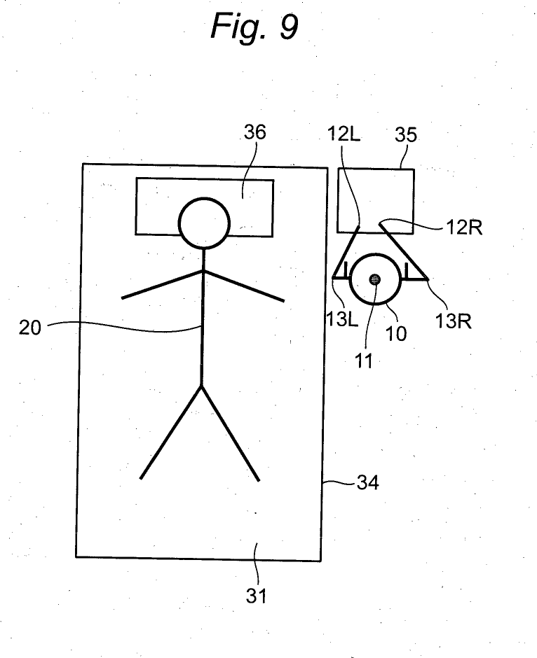

“Any object in the patient's surroundings” refers to, for example, a bed frame 34, a bedside cabinet 35, and a pillow 36 shown in FIG. 9. When the health-care worker touches any object in the patient's surroundings, the wrist of the health-care worker is positioned near the object in the patient's surroundings.

For example, as shown in FIG. 9, when the subject sensing device 2 senses that the right wrist 12R or the left wrist 12L is disposed in the range spanning by a prescribed distance (e.g., 10 cm) or less from the bedside cabinet 35 sensed by the object sensing device 1, it is estimated that the subject 10 has touched the bedside cabinet 35.

The storage unit 4 previously stores the combination of the sensing information pieces of the sensing device 1 to 3 at this time. The counter 5 counts the number of times the combination of the sensing information pieces of the sensing device 1 to 3 achieved the combination stored in the storage unit 4. Thus, the hand hygiene opportunity “after touching any object in the patient's surroundings” can be measured.

As described above, with the hand hygiene moment measuring system according to the first embodiment, all the five moments can be automatically sensed. Therefore, the burden incurred by measurement of the hand hygiene opportunities can be largely reduced, and such measurement can be carried out highly precisely.

Note that, in the foregoing, while examples of specific movements at five moments have been described, there exist other specific movements. For example, a bed bath, which is the movement of a health-care worker wiping the body of a patient, requires the health-care worker to take a hand hygiene action, because the hands of the health-care worker may carry germs after giving the bed bath. That is, the bed bath corresponds to the hand hygiene opportunity “after touching a patient”. When carrying out a bed bath, the health-care worker is normally at the area near the bed, holding the patient with one hand while widely swinging the other hand. For example, when the movement sensing device 3 senses that the left wrist 12L is in the still state and the right wrist 12R is swinging, it can be estimated that a bed bath is given to the patient. In this manner, substantially every specific movement at five moments can be sensed by the sensing device 1 to 3.

Note that the present invention is not limited to the embodiment described above, and can be practiced in various manners. For example, the combinations of the sensing information pieces of the sensing device 1 to 3 indicative of the hand hygiene opportunities are not limited to those described above, and can be changed as appropriate.

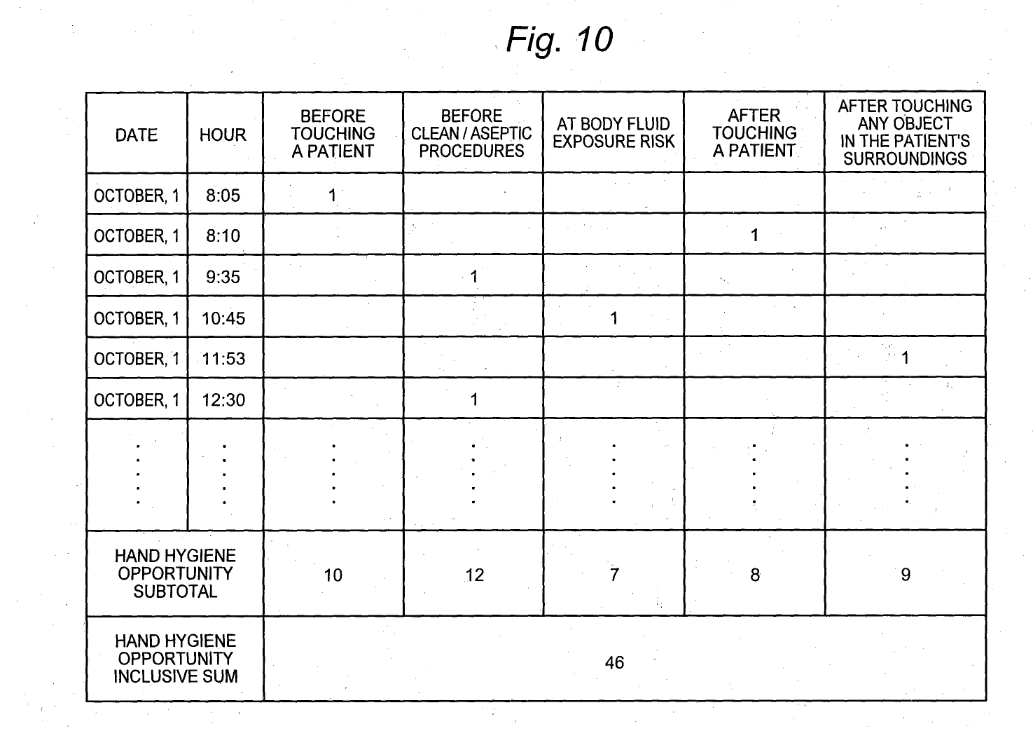

Further, the storage unit 4 preferably stores particular combinations corresponding to hand hygiene opportunities as being classified into a plurality of groups, such as five moments. Further, the counter 5 preferably counts the number of times the combinations of the sensing information of the object sensing device 1, the sensing information of the subject sensing device 2, and the sensing information of the movement sensing device 3 achieved the combinations stored in the storage unit 4 with respect to each of the groups. Thus, for example, it becomes possible to cause the notifying unit 6 to display information such as shown in FIG. 10, and usability can be improved.

Note that, another possible method for measuring the hand hygiene opportunities may be, for example, shooting a video in the hospital room, and measuring the hand hygiene opportunities based on the shot video. However, in this case, privacy of the patient may not be ensured.

Therefore, it is preferable that the subject sensing device 2 and the movement sensing device 3 recognize the health-care worker and the patient as distinguished from each other, and that the storage unit 4 does not store sensing information on the patient (e.g., information specifying the face of the patient or the like). Further, when the notifying unit 6 displays the patient 20 as an image, the image is preferably shown in a manner in which the patient 20 cannot be identified, i.e., as the stickman shown in FIG. 2. Thus, privacy of the patient 20 can be ensured.

Note that, in order to distinguish between the health-care worker and the patient, the hand hygiene moment measuring system according to the first embodiment may further include, for example, a color sensor. Since health-care workers often dress in white, for example, when the color sensor recognizes a subject with a high white color proportion, the subject can be recognized as a health-care worker. Further, it is also possible to put an identification tag to a health-care worker, such that the subject can be recognized as a health-care worker by the tag being read. Further, for example, the voice recognition sensor may be further included. By the voice recognition sensor sensing the voice uttered by a health-care worker, the subject may be identified as a health-care worker.

Further, in order to carry out measurement of the hand hygiene opportunities further highly precisely, the hand hygiene moment measuring system according to the first embodiment may further include a speech recognition sensor. Thus, for example, even when any hand hygiene opportunity that cannot be sensed by the sensing device 1 to 3 has occurred, by extracting and recognizing specific words such as “intravenous drip”, “diaper”, “change”, and the like from the speech of the subject, the hand hygiene opportunities can be counted.

### Second Embodiment

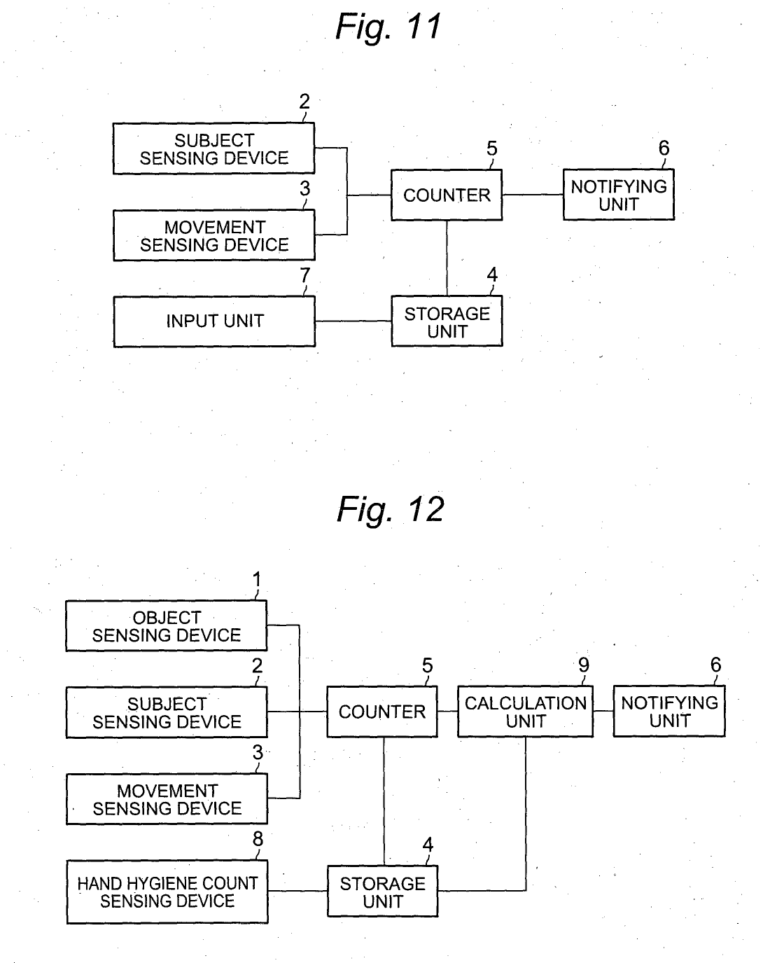

With reference to FIG. 11, a description will be given of the overall structure of a hand hygiene moment measuring system according to a second embodiment of the present invention. FIG. 11 is a block diagram of the hand hygiene moment measuring system according to the second embodiment. The hand hygiene moment measuring system according to the second embodiment is different from the hand hygiene moment measuring system according to the first embodiment in including an input unit 7, in place of the object sensing device 1.

The position of an object such as a bed or a bedside cabinet is generally fixed. Accordingly, the position of such objects can be previously input by the user without the necessity of having the object sensing device 1 automatically perform sensing.

Therefore, the input unit 7 is structured to be capable of receiving inputs of object position information. The object position information input into the input unit 7 is stored in the storage unit 4, for example. As the input unit 7, for example, any device built in a personal computer can be used.

The counter 5 counts the number of times the combinations of the sensing information of the object sensing device 1, the sensing information of the subject sensing device 2, and the object position information input into the input unit 7 achieved the combinations stored in the storage unit 4.

With the hand hygiene moment measuring system according to the second embodiment, since all the five moments can be automatically sensed, the burden incurred by measurement of hand hygiene opportunities can be largely reduced, and such measurement can be carried out highly precisely.

### Third Embodiment

With reference to FIG. 12, a description will be given of the overall structure of a hand hygiene moment measuring system according to a third embodiment of the present invention. FIG. 12 is a block diagram of the hand hygiene moment measuring system according to the third embodiment. The hand hygiene moment measuring system according to the third embodiment is different from the hand hygiene moment measuring system according to the first embodiment in further including a hand hygiene count sensing device 8 and a calculation unit 9.

The hand hygiene count sensing device 8 senses the number of taken hand hygiene actions. As the hand hygiene count sensing device 8, any known apparatus such as the one disclosed in the aforementioned Patent Literature 1 can be used. The number of taken hand hygiene actions sensed by the hand hygiene count sensing device 8 is stored in the storage unit 4.

The calculation unit 9 calculates the hand hygiene compliance rate based on the number of taken hand hygiene actions stored in the storage unit 4 and the number of hand hygiene opportunities counted by the counter 5. The compliance rate calculated by the calculation unit 9 is visually or aurally notified by the notifying unit 6.

According to the third embodiment of the present invention, since the notice about the compliance rate of the hand hygiene opportunities is provided, usability can be further improved.

By properly combining arbitrary embodiments, of the aforementioned various embodiments, the effects owned by each of them can be made effectual.

Although the present invention has been fully described in connection with the preferred embodiments thereof with reference to the accompanying drawings, it is to be noted that various changes and modifications are apparent to those skilled in the art. Such changes and modifications are to be understand as included within the scope of the present invention as defined by the appended claims unless they depart therefrom.

The entire disclose of Japanese Patent Application No. 2013-018442 filed on Feb. 1, 2013, including specification, drawings, and claims is incorporated herein by reference in the entirety.

### INDUSTRIAL APPLICABILITY

The hand hygiene moment measuring system of the present invention reduces the burden incurred by measurement of hand hygiene opportunities and carries out such measurement highly precisely. Accordingly, it is particularly useful for measuring hand hygiene opportunities at a site such as an intensive care unit where HAIs must be prevented.

### REFERENCE SIGNS LIST

1 object sensing device
2 subject sensing device
3 movement sensing device
4 storage unit
5 counter
6 notifying unit
7 input unit
8 hand hygiene count sensing device
9 calculation unit
10 subject
11 trunk
12R right wrist
12L left wrist
13R right shoulder
13L left shoulder
20 patient
31 bed
32 intravenous drip stand
33 region
34 bed frame
35 bedside cabinet
36 pillow

1. A hand hygiene moment measuring system comprising:
a subject sensing device to sense a subject's position and posture;
a movement sensing device to sense a subject's movement;
a storage unit to store particular combinations corresponding to hand hygiene opportunities; and
a counter to count a number of times combinations of a sensing information of the subject sensing device, a sensing information of the movement sensing device, and an object position information indicating a position of an object, achieved the combinations stored in the storage unit.

2. The hand hygiene moment measuring system according to claim 1, further comprising
an object sensing device to sense the position of the object, wherein
the counter counts a number of times combinations of the sensing information of the subject sensing device, the sensing information of the movement sensing device, and a sensing information of the object sensing device, achieved the combinations stored in the storage unit.

3. The hand hygiene moment measuring system according to claim 1, further comprising
an input unit to receive inputs of the object position information, wherein
the storage unit stores the object position information input into the input unit, and
the counter counts a number of times combinations of the sensing information of the subject sensing device, the sensing information of the movement sensing device, and the object position information stored in the storage unit, achieved the combinations stored in the storage unit.

4. The hand hygiene moment measuring system according to claim 1, wherein
the storage unit stores particular combinations corresponding to hand hygiene opportunities as being classified into a plurality of groups, and
the counter counts a number of times combinations of the sensing information of the subject sensing device, the sensing information of the movement sensing device, and the object position information stored in the storage unit, achieved the combinations stored in the storage unit with respect to each of the groups.

5. The hand hygiene moment measuring system according to claim 1, further comprising a notifying unit to notify about the number of times counted by the counter.

6. The hand hygiene moment measuring system according to claim 1, further comprising
a calculation unit to calculate the hand hygiene compliance rate based on the number of times counted by the counter, and
a notifying unit to notify the compliance rate calculated by the calculation unit.
